(19) United States
(12) Patent Application Publication
(10) Pub. No.: US 2024/0377395 A1
(43) Pub. Date: Nov. 14, 2024

(54) AGGLUTINATING AGENTS, DEVICES AND METHODS FOR WHOLE BLOOD ASSAYS

(71) Applicant: Quidel Corporation, San Diego, CA (US)

(72) Inventors: Jason J. Sun, San Diego, CA (US); Jason McClure, San Diego, CA (US); Kevin S. Richardson, San Diego, CA (US)

(21) Appl. No.: 18/690,631

(22) PCT Filed: Sep. 9, 2022
(86) PCT No.: PCT/US2022/076220

§ 371 Date: Mar. 8, 2024

Related U.S. Application Data
(60) Provisional application No. 63/242,754, filed on Sep. 10, 2021.

(51) Int. Cl. G01N 33/569; G01N 33/543; G01N 33/58

(52) U.S. Cl. CPC G01N 33/56966; G01N 33/543; G01N 33/58

(57) ABSTRACT

Devices, methods and kits including a means for capturing red blood cells are provided. Such means include physical means for trapping red blood cells as well as agglutination agents, including hemagglutination agents, for retaining red blood cells in certain regions of the devices described herein. The physical means and agglutination agents provide separation of red blood cells from whole blood samples. Analysis of separated and/or agglutinated whole blood samples for the presence or absence of analytes of interest can be performed without interference from red blood cells. Devices, such as lateral flow assays with sample receiving zones comprising physical means and/or agglutination agents are also described. Certain agglutination agents include lectins and antibodies. such as red blood cell binding antibodies.

### CROSS-REFERENCE TO RELATED APPLICATIONS

This application claims the benefit of U.S. Provisional Application No. 63/242,754, filed Sep. 10, 2021, which is incorporated by reference herein.

### TECHNICAL FIELD

The present disclosure generally relates to devices and methods comprising a means for capturing red blood cells, such as physical means and agglutination agents, including hemagglutination agents, for immunoassays, such as lateral flow assays for analysis of whole blood.

### BACKGROUND

Various devices, assays, methods and techniques can be used to analyze body fluids, such as whole blood, for the presence or absence of a particular analyte of interest. For example, tests are available to detect glucose, uric acid or protein in urine, or to detect pathogens, antibodies, glucose, triglycerides, potassium ion or cholesterol in blood. Devices and methods to process whole blood by removing red blood cells prior to testing are used, for example, to separate the fluid sample into separate portions or fractions, such as a red blood cell fraction and a plasma fraction. For example, cellular components of whole blood can be separated from the plasma or serum for use in an assay to test for a particular analyte of interest. The resulting plasma or serum can be examined for accurate detection of the analyte of interest without interference from the red blood cells present in the whole blood sample.

The cellular components of whole blood, and especially the red blood cells, are the primary interfering substances in assays, such as devices comprising lateral flow immunoassays or strip tests, for an analyte of interest present in whole blood. Many blood tests are chromogenic, wherein an analyte present in whole blood interacts with a particular reagent either to form a uniquely-colored complex or derivative as a quantitative or qualitative indication of the presence or absence of the analyte, or to form a colored complex or derivative of variable color intensity as a quantitative indication of the presence of the constituent. The deep red color of the whole blood sample substantially interferes with these chromogenic tests, and therefore the highly-colored red blood cells usually are separated from the plasma or serum before the blood sample is assayed for a particular analyte of interest. The presence of red blood cells also can interfere with various nonchromogenic blood assays, whereby the assay results are either inconsistent or, if consistent, are inaccurate.

### BRIEF SUMMARY

The subject technology is illustrated, for example, according to various aspects described below.

According to some embodiments a device for detection of an analyte of interest in a whole blood sample is provided. In some aspects, the devices provided herein comprise an immunoassay, such as a lateral flow assay or strip test. In some aspects, devices comprise a sample receiving zone comprising a means for capturing red blood cells. In some embodiments, the means is a physical means, such as a net or mesh present in the sample pad of a lateral flow assay or strip test, wherein the net/mesh physically interacts with red blood cells to retain them in the sample pad without lysis of the red blood cells. In some embodiments, the sample pad may also comprise a non-lytic hemagglutination agent, wherein the hemagglutination agent captures red blood cells from an applied whole blood sample in the sample receiving zone without lysis of the red blood cells.

In other aspects, devices comprise a labeling zone comprising a means for specifically labeling the analyte of interest. In other aspects, the devices also include a capture zone comprising means for specifically binding and immobilizing the labeled analyte of interest. In some aspects, the devices comprise a sample receiving zone, labeling zone and capture zone arranged in a liquid flow path.

In some embodiments, sample receiving zones, including sample pads, comprise a means for physical interaction with red blood cells comprising a structure such as a net, mesh, screen, or lattice with appropriate gap sizes for physically retaining and/or trapping red blood cells in the sample pad of the lateral flow strip test devices described herein, wherein red blood cells are trapped and/or retained in the sample pad by interaction with the physical structure.

In some embodiments, the hemagglutination agent is an antibody, such as a monoclonal or polyclonal antibody. In some embodiments, the antibodies bind red blood cells, such as human red blood cells. In some embodiments, the antibodies bind H antigen of human red blood cells, and therefore bind essentially all human red blood cells regardless of ABO type. In some embodiments, the antibodies are biotinylated.

In some embodiments, the hemagglutination agents, such as antibodies, may be self-immobilized in a device, such as in the sample receiving zone of a device comprising an immunoassay, such as a lateral flow assay or strip test.

In some embodiments, the device may comprise an immunoassay, such as a lateral flow assay with a sample receiving zone (i.e., sample pad), wherein the sample receiving zone comprises streptavidin. In some embodiment, the streptavidin is associated with or on particles immobilized in a sample receiving zone of a lateral flow immunoassay.

In some embodiments, the hemagglutination agent is a lectin. In some aspects, the lectin is any lectin, including but not limited to, wheat germ agglutinin, Ulex europaeus agglutinin I, Pisum sativum agglutinin, Lens culinaris agglutin, or Phaseolus vulgaris erythroagglutinin. In some embodiments, a lectin may be self-immobilized in the device, such as in a sample receiving zone of a device comprising an immunoassay, such as a lateral flow assay or strip test. In some embodiments, the lectins are biotinylated.

In some embodiments, a means for specifically labeling an analyte of interest is a detectable antibody, such as a detectable monoclonal antibody or detectable polyclonal antibody.

In some embodiments, a means for specifically labeling an analyte of interest is a detectable antigen, such as a protein, peptide, macromolecule, or small molecule.

In some embodiments, a means for specifically binding and immobilizing a labeled analyte of interest is an antibody, such as an immobilized antibody. In some embodiments, the immobilized antibody is a monoclonal antibody or a polyclonal antibody.

In some embodiments, a means for specifically binding and immobilizing an analyte of interest is an antigen, such as a immobilized antigen. In some embodiments, the immobilized antigen is an immobilized protein, immobilized peptide, immobilized macromolecule, or immobilized small molecule.

In another embodiment, methods for detecting the presence or absence of an analyte of interest in a whole blood sample are provided. In some embodiments, the methods comprise providing a device as described herein, placing a whole blood sample on the device, wherein red blood cells are retained in the sample receiving zone; and determining the presence or absence of labeled analyte immobilized at the capture zone. In some embodiments, the methods also include providing an instrument for collecting the whole blood sample; and collecting a whole blood sample on the instrument for addition to a device, such as a sample receiving zone of a device comprising a lateral flow immunoassay.

In another embodiment, a kit comprising a device as described herein, an instrument for collecting a whole blood sample; and instructions for use is provided. In some aspects, the instrument for collecting a whole blood sample comprises a capillary tube.

In addition to the exemplary aspects and embodiments described above, further aspects and embodiments will become apparent by reference to the drawings and by study of the following descriptions.

Additional embodiments of the present devices, assays, methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment described herein. Additional aspects and advantages of the present disclosure are set forth in the following description and claims, particularly when considered in conjunction with the accompanying examples and drawings.

### BRIEF DESCRIPTION OF THE DRAWINGS

FIG. 1 illustrates human red blood cell agglutination testing with various lectins as the agglutination agent.

FIG. 2 illustrates human red blood cell agglutination testing with an anti-human red blood cell antibody as the agglutination agent.

FIG. 3A illustrates human red blood cell trapping in a lateral flow immunoassay with a sample receiving zone comprising control condition 1. In this configuration sample pad treatments included blank control without any antibody, and retention of red blood cells in the sample pad was achieved by physical means such as the red blood cells being trapped in a net-like mesh of the sample pad.

FIG. 3B illustrates human red blood cell trapping in a lateral flow immunoassay with a sample receiving zone comprising hemagglutination agents of condition 2. In this configuration sample pad treatment included mAb H101, and retention of red blood cells in the sample pad was achieved by agglutination of red blood cells by the antibody in addition to physical means such as the agglutinated red blood cells being trapped in the net-like mesh of the sample pad.

FIG. 3C illustrates human red blood cell trapping in a lateral flow immunoassay with a sample receiving zone comprising hemagglutination agents of condition 3. In this configuration, sample pad treatments included mAb H101 with streptavidin, and retention of red blood cells in the sample pad was achieved by agglutination of red blood cells by the antibody and/or streptavidin in addition to physical means such as the agglutinated red blood cells being trapped in the net-like mesh of the sample pad.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

The various devices, assays, methods and techniques provided herein can be used to analyze body fluids, such as whole blood, for the presence or absence of a particular analyte of interest without interference from red blood cells. The technology provided herein, such as agglutinating agents and devices and methods comprising the same, provide analysis of whole blood samples without red blood cell interference and without the need for extra, expensive, cumbersome, complicated, or time consuming steps.

Conventionally, plasma or serum can be separated from the cellular material of whole blood by centrifugation or by clotting. The cellular material collects at the bottom of the centrifuge or sample tube and the supernatant plasma or serum is decanted. Accordingly, the interfering cellular components of whole blood are removed such that a substantial background interference, such as red blood cell interference, is avoided. However, the centrifuge method requires a large blood sample, a long centrifuge time, and appropriate equipment. Furthermore, the centrifuge method requires several manipulative steps. The clotting method of obtaining serum also requires a long time (30-60 minutes) for clotting to occur and centrifugation may be required after clotting occurs.

Other techniques can involve test strips for lateral flow of samples for analysis. However, in some designs, red blood cells form aggregates or blockages within the chemical strip that disrupts the lateral flow and/or results in discoloration of the test strip making it difficult for results to be interpreted. Such blockages often produce inaccurate and/or invalid results.

Lateral flow immunoassay devices described herein include a sample receiving zone comprising a sample pad that is configured such that the sample pad physically interacts with red-blood cells in a manner that causes red blood cells to be retained or trapped within the pad material. The sample pad comprises a means for physical interaction with red blood cells in a manner which holds, retains, traps, and/or otherwise slows the flow of red blood cells from the sample pad to downstream portions of the lateral flow strip test device. For example, sample pads may include a means for physical interaction with red blood cells comprising a structure such as a net, mesh, screen, or lattice with appropriate gap sizes for physically retaining and/or trapping red blood cells in the sample pad of the lateral flow strip test devices described herein.

In addition, some lateral flow immunoassay devices and methods described herein include agglutinating agents, that efficiently separate and accurately assay whole blood samples without the need for additional processing steps to be performed by the end user. The provided agents, devices, and methods can shield a user, such as an at home tester or lab technician, from unnecessary contact with a blood sample, avoid time delays, and yield accurate and reproducible results.

Techniques based on the present disclosure can allow the safe, accurate, and economical assay of a whole blood sample for a particular soluble component, such as an analyte of interest, to achieve essentially total separation of the red blood cells of whole blood from the plasma or serum. It will be appreciated that the systems, devices, methods and kits described herein are contemplated for use with a variety of fluid samples, and whilst the description herein uses blood as a model sample, the systems and devices and methods may also be used with other fluid samples.

### Agglutination Agents

Embodiments described herein comprise an agglutination agent, such as a hemagglutination agent, to aggregate red blood cells and/or cause red blood cells to clump together. As used herein, “agglutination” refers to the clumping together of cells and “hemagglutination” refers to the clumping together of red blood cells. In some instances, “agglutination” and “hemagglutination” are related terms that may be used interchangeably. For example, agglutinated or hemagglutinated red blood cells are clumped together by the agents described herein and can then be trapped or removed so that they do not interfere in analysis of the remaining sample for the presence of absence of an analyte of interest.

In some embodiments, the agglutination agents are “non-lytic” meaning they do not cause cell lysis or disintegration of a cell by rupture of the cell wall or membrane. Instead, in some embodiments the agglutination agents provide hemagglutination of intact red blood cells without rupture or lysis of the agglutinated cells. This feature results in trapping or retention of all red blood cell components by agglutination of the intact red blood cells so that internal cellular components, including heme groups, are not released and therefore cannot interfere in subsequent and/or downstream sample analysis because they are retained in the agglutinated and trapped, intact red blood cells.

### Lectins

In some embodiments, the agglutinating agent is a lectin. Lectins have had a long history as medical diagnostic reagents and tools for many applications, with good affinity and multi-valency for specific binding to complex carbohydrates, such as surface antigens on red blood cells. Lectins are typically composed of four subunits, i.e., tetra-valent for efficient agglutination/trapping of red blood cells. Accordingly, certain lectins can be used to greatly enhance the separation of plasma fluid from various red blood cells. Hence, devices and methods comprising lectins specific for all red blood cells, such as all types of human red blood cells, may be used for the separation, trapping and retention of human red blood cells from whole blood samples of any blood type. Suitable lectins include, but are not limited, those included in Table 1, which are suitable for agglutinating and trapping human red blood cells of all blood types (ABO). Moreover, in some embodiments, biotinylated lectins may be used in combination with streptavidin (SA), to provide improved hemagglutinating activity for more efficient and effective binding, trapping and retention of human red blood cells, such as trapping red blood cells in a lectin impregnated sample pad of a sample receiving zone of a lateral flow device as provided herein.

TABLE 1
Example Lectins With Reactivity To Human Red Blood Cells Of All Blood Types

| | Lectin | Carbohydrate | A | B | AB | O |
|---|---|---|---|---|---|---|
| Systematic name | name | specificity | specific | specific | specific | specific |
| Canavalia ensiformis | Con A | α-D-Man; α-D-Glc | + | + | + | + |
| Glycine Max | SBA | α, β Gal; α, β Gal NAc | ++ | + | + | + |
| Pisum sativum | PSA | α-D-Man | + | + | + | + |
| Phaseolous vulgaris | PHA-E | Mannose oligosaccharide | + | + | + | + |
| Solanum Tuberosum | STL | (β-D-GlcNAc)2-5 | + | + | + | + |
| Ulex Europaeous | UEL | α-L-Fuc | + | + | + | ++ |
| Triticum vulgaris | WGA | β-D-GlcNAc; Neu5Ac | + | + | + | + |

In some embodiments, lectins can be recombinantly expressed for scale up production. Recombinant expression of lectins in native form or with tandem repeats has been systematically explored. In some embodiments, the lectins may be biotinylated for use in conjunction with devices and methods that include streptavidin and utilize the biotin-streptavidin advantageously. In some embodiments, the lectins may include tandem repeats that can increase the efficiency of lectin production on a large scale in a bacterial expression system. (Hwang et al., Biomolecules 2018, 8, 146). Further fusion technologies have been widely used for proteins and can be applicable for producing lectins with better trapping efficiency.

In some embodiments, lectins may be conjugated to bead nanoparticles. In such situations, the lectin conjugated beads or nanoparticles provide efficient agglutination agents since they can agglutinate and trap/retain numerous blood cells on multi-valent surfaces. In some embodiments, recombinant fusion technology, allows lectins with a fusion tag to be directly attached to nanoparticles with higher efficiency and low cost for production and use as agglutination agents.

### Agglutinating Antibodies

In other embodiments, the agglutinating agent is an antibody, such as an antibody with binding specificity for red blood cells. Human ABO blood type discrimination is based on the presence of the non-reducing aGal and aGalNAc terminals, whereas aFuc is the determinant of the H(O) blood group. Accordingly, these carbohydrate antigens provide desirable targets for agglutination agent binding, such as lectins and/or antibodies. Antibodies are bi-valent in nature and therefore, antibodies with red blood cell specificity provide effective agglutination agents for retaining/trapping red blood cells.

Polyclonal and/or monoclonal antibodies with red blood cell binding affinity may be used as an agglutination agent. For example, polyclonal or monoclonal antibodies with binding specificity for the H antigen of human red blood cells may be used as agglutination agents according to certain embodiments. In some embodiments, a monoclonal antibody against the H(O) blood group is provided as an agglutination agent. Such monoclonal antibodies demonstrate desirable hemagglutinating activity with pooled human red blood cells (hRBC) of mixed blood types. Additionally, in some embodiments, biotinylated antibodies may be used in combination with streptavidin (SA), to provide improved hemagglutinating activity for more efficient and effective binding, trapping and retention of human red blood cells, such as trapping red blood cells in an antibody impregnated sample pad of a sample receiving zone of a lateral flow device as provided herein.

In some embodiments, antibodies can be recombinantly expressed for scale up production. Recombinant expression of antibodies in native form or with tandem repeats has also been explored. In some embodiments, red blood cell specific antibodies may be biotinylated for use in conjunction with devices and methods that include streptavidin and utilize the biotin-streptavidin advantageously. In some embodiments, the provided antibodies may also include tandem repeats that can increase the efficiency of antibody production on a large scale in a bacterial expression system. (Hwang et al., Biomolecules, 2018, 8, 146). Further, fusion technologies have been widely used for proteins and antibodies, and can be applicable for producing antibodies with improved red blood cell agglutinating and trapping efficiency.

In some embodiments, antibodies may be conjugated to bead nanoparticles. In such situations, the antibody conjugated beads or nanoparticles provide efficient agglutination agents since they can agglutinate and trap/retain numerous blood cells on multi-valent surfaces. In some embodiments, recombinant fusion technology allows antibodies with a fusion tag to be directly attached to nanoparticles with high efficiency and low cost for production and use as agglutination agents.

### Lateral Flow Devices, Methods and Kits With Agglutination Agent(s)

### Devices

In some embodiments, the agglutinating agents are present in or on a device, such as a lateral flow immunoassay or strip test. As used herein, “sample receiving zone” refers to a portion of an assay or device, such as a lateral flow device or strip test, that is configured for sample addition. In some embodiments, the sample receiving zone may comprise an agglutination agent or comprise a sample pad that comprises or is impregnated with said agglutination agent or agents.

As used herein, “label zone” refers to a portion of an assay or device, such as a lateral flow device or strip test, that is configured to comprise mobilizable reagents for labeling an analyte of interest, such as antibodies or antigens that bind an analyte of interest. In some embodiments, the mobilizable binding agents are specific for an analyte of interest and detectable, such as optically or visually detectable.

As used herein, “capture zone” refers to a portion of an assay or device, such as a lateral flow device or strip test, that is configured to comprise immobilized reagents with specific binding for capturing a labeled analyte of interest, such as capture of mobilizable, detectable agents conjugated to analytes of interest. In some embodiments, the capture zone may comprise immobilized antibodies or antigens that bind an analyte of interest and a conjugate of the analyte of interest with a mobilizable detectable agent. In some embodiments, the agents of the capture zone result in accumulation of labeled analyte of interest, if present in the analyzed sample, at a test line that can be visually or optically inspected and/or detected.

As used herein, “liquid flow path” refers to a path on an assay, such as a lateral flow immunoassay, where an applied sample flows in an upstream to downstream direction. Accordingly, in some embodiments, lateral flow devices include an upstream sample receiving zone, a label zone downstream of the sample receiving zone and a capture zone downstream of the label zone, all present in the same liquid flow path so that an added sample will flow from the sample receiving zone, through the label zone and into the capture zone.

In some embodiments, the lateral flow devices described herein include a sample receiving zone comprising a sample pad that is impregnated with an agglutinating agent, such as a lectin or red blood cell specific antibody, such that the sample pad and agglutinating agents interact with red-blood cells in a manner that causes red blood cells to be retained or trapped, chemically and/or physically, within the pad material. Systems, methods, and kits can incorporate a device as described herein and a blood capillary tube, such as a capillary with a volume monitoring mechanism, for sample collection. Embodiments can facilitate removal of red-blood cells from a venipuncture or finger stick whole blood sample, after the whole blood sample is added to a sample receiving zone of a lateral flow immunoassay, and while the sample moves through the lateral flow device. Accordingly, plasma and buffer solution move through the test strip for analysis while the red blood cells are agglutinated and trapped/retained in the sample pad of the sample receiving zone. The agglutinating agents and devices and methods including the same, can simplify work- flow for the end-user and can be applied universally across assays that utilize a whole blood sample for detection of an analyte of interest, and particularly in lateral flow immunoassay strip tests for analysis of whole blood for an analyte of interest.

In some embodiments, the lateral flow devices include a sample receiving zone comprising a sample pad that is configured such that the sample pad physically interacts with red-blood cells in a manner that causes red blood cells to be retained or trapped within the pad material. In such embodiments, the sample pad comprises a means for physical interaction with red blood cells in a manner which holds, retains, traps, and/or otherwise slows the flow of red blood cells from the sample pad to downstream portions of the lateral flow strip test device. For example, sample pads may include a means for physical interaction with red blood cells comprising a structure such as a net, mesh, screen, or lattice with appropriate gap sizes for physically retaining and/or trapping red blood cells in the sample pad of the lateral flow strip test devices.

In other embodiments, agglutination and trapping of the red-blood cells in the sample pad of a sample receiving zone in a lateral flow device improves the efficiency of plasma separation. Specifically red blood cell capture in the agglutination agent impregnated sample pad requires only addition of a whole blood sample in buffer to the sample receiving zone and results in red blood cells being agglutinated, trapped and retained in the sample pad. This prevents red blood cells from entering the sample label zone or test zone of the lateral flow immunoassay and, therefore, prevents red blood cells from interfering with production or interpretation of strip test results. Agglutination of the red blood cells in an agglutination agent impregnated sample pad traps and retains red blood cells in the sample pad without red blood cell lysis or leaking from the sample pad into the downstream components and areas of the lateral flow immunoassay device. This prevents red blood cells from interfering with downstream agents for labeling and immobilization of an analyte of interest that may also be present in the tested whole blood sample.

### Kits

According to embodiments, kits using the devices comprising agglutinating agents are also provided. Instructions and additional components, such as a sample collection means, in addition to the agglutination agents, devices and methods described herein are also provided. In some embodiments, a means or device for collection of a whole blood sample may comprise a capillary tube.

According to embodiments, a kit of parts can include a device as described herein and additional components for sample collection, sample dilution (buffer), and/or use of the device. Directions for use (“DFU”) can be included with the kit. One or more collection devices may be included and disposable as intended for one time use.

### Methods

According to other embodiments, methods that employ components of the devices or kits, as described herein, are provided. The methods, in some embodiments, achieve analysis of a fluid sample, such as analysis of a whole blood sample for the presence or absence of an analyte of interest, without interference from certain components of the whole blood sample, such as red blood cells. Accordingly, methods for detecting the presence or absence of an analyte of interest in a whole blood sample comprise, in embodiments, providing a device as described herein, placing a whole blood sample on the device, such as on or in a sample receiving zone comprising an agglutinating agent impregnated sample pad, wherein red blood cells are retained in the sample receiving zone, and the presence or absence of labeled analyte immobilized at the capture zone is visualized.

A method can employ components of the system or kit, as described herein, to achieve agglutination of red cells present in a whole blood sample. According to some embodiments a sample, such as whole blood, can be collected, applied to a device and agglutinated, such as agglutinated by agglutination agents present in a sample pad of a sample receiving zone of a lateral flow immunoassay device, wherein agglutinated red blood cells are trapped/retained in the sample pad while the remainder of the sample flows downstream for analysis for the presence of the analyte of interest by interactions of the sample with components present in the label and test zones.

### EXAMPLES

The following examples are illustrative in nature and are in no way intended to be limiting.

### Example 1: Human Red Blood Cell Agglutination Testing With Various Lectins

A study was performed to evaluate various lectins ability to agglutinate human red blood cells. The tested lectins were biotinylated versions commercially available from Vector Labs having sugar specificity for mannose, galactose, fucose, N-acetylgalactosamine, and N-acetylglucosamine. Specifically, the tested lectins include biotinylated versions of concanavalin A (Con A), glycine max (soybean) agglutinin (SBA), triticum vulgaris (wheat germ) agglutinin (WGA), ulex europaeus agglutinin I (UEA I), arachis hypogaea (peanut) agglutinin (PNA), pisum sativum agglutinin (PSA), lens culinaris agglutinin (LCA), phaseolus vulgaris erythroagglutinin (PHA-E).

Agglutination assays were performed in round bottom 96-well plates and using an orbital shaker/incubator for mixing and temperature control. The lectins were analyzed for ability to agglutinate pooled human red blood cells suspended in 10% PBS solution (Rockland #R407-0050). Agglutination assays were performed for each lectin with, and without addition of streptavidin (SA).

Serially diluted lectin solutions were prepared with and without streptavidin and 95 μL of the serially diluted lectin solution was pipetted into a well of a 96-well plate. Next, 5 μL of the pooled human red blood cell suspension was added to the lectin solutions. The solutions were thoroughly mixed by pipetting and on an orbital shaker. The lectin/human red blood cell mixtures were incubated for 1 hour at room temperature. After the 1 hour incubation period, agglutination results were visualized.

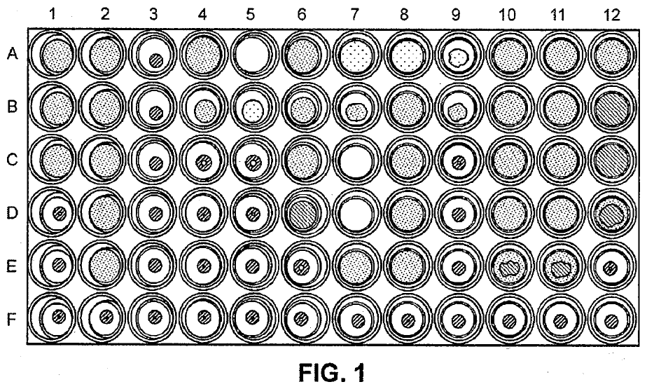

Results of the agglutination assay for some lectins are shown in FIG. 1 as follows: Column 1: WGA without SA, Column 2: UEA without SA, Column 3: PNA without SA, Column 4: PSA without SA, Column 5: LCA without SA, Column 6: PHA-E without SA, Column 7: WGA with SA, Column 8: UEA with SA, Column 9: PNA with SA, Column 10: PSA with SA, Column 11: LCA with SA, Column 12: PHA-E with SA. Lectin concentrations in the test wells after serial dilution are as follows: Row A: 100 μg/mL, Row B: 30 μg/mL, Row C: 10 μg/mL, Row D: 3 μg/mL Row E: 1 μg/mL Row F: 0 g/mL.

Based on agglutination assay results, half maximal effective concentrations (EC50) were calculated for tested lectins as shown in Table 2.

TABLE 2
Lectin EC50 values

| Lectin | EC50, ug/mL (w/o SA) | EC50, ug/mL (with SA) |
|---|---|---|
| ConA | ~100 | 30 |
| SBA | 30 | 30 |
| WGA | 10 | 1 |
| UEA-I | 1 | <1 |
| PNA | ~100 | 30 |
| PSA | 30 | 1 |
| LCA | 30 | 1 |
| PHA-E | 10 | 3 |

This data demonstrates lectins with various sugar specificities provide hemagglutinating activity of pooled human red blood cells (hRBC). In addition, some biotinylated lectins, such as WGA, UEA-I, PSA, LCA and PHA-E, when tested in combination with streptavidin, provide improved hemagglutination of pooled human red blood cells as demonstrated by lower EC50 values.

### Example 2: Human Red Blood Cell Agglutination Testing With Lectins And Anti-Human Red Blood Cell Antibody

A study was performed to evaluate various lectins and an anti-human red blood cell antibody abilities to agglutinate human red blood cells. The tested lectins were biotinylated versions commercially available from Vector Labs having sugar specificity for mannose, and galactose. Specifically, the tested lectins include biotinylated versions of concanavalin A (Con A) and Glycine max (soybean) agglutinin (SBA). The tested anti-human red blood cell antibody is biotinylated monoclonal anti-hRBC H101, engineered with Avitag, and in vitro biotinylated. Anti-hRBC mAb H101 was produced recombinantly to exhibit specificity for H antigen of hRBCs.

Agglutination assays were performed in round bottom 96-well plates and using an orbital shaker/incubator for mixing and temperature control. Anti-hRBC mAb H101 and the lectins were analyzed for ability to agglutinate pooled human red blood cells suspended in 10% PBS solution (Rockland #R407-0050). Agglutination assays were performed for each agglutination agent (Ab and lectins) with, and without addition of streptavidin (SA).

Serially diluted antibody and lectin solutions were prepared with and without streptavidin and 95 μL of the serially diluted antibody/lectin solution was pipetted into a well of a 96-well plate. Next, 5 μL of the pooled human red blood cell suspension was added to the lectin solutions. The solutions were thoroughly mixed by pipetting and on an orbital shaker The agglutination agent/human red blood cell mixtures were incubated for 1 hour at room temperature. After the 1 hour incubation period, agglutination results were visualized.

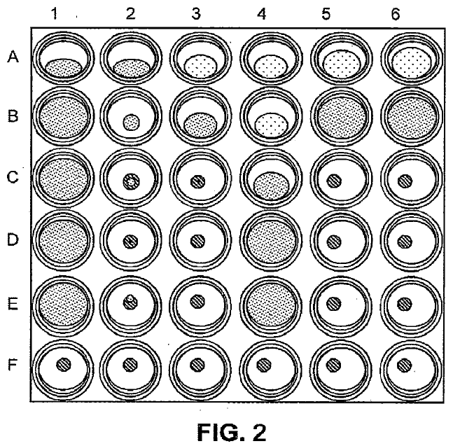

Results of the agglutination assay for some lectins are shown in FIG. 2 as follows: Column 1: mAb H101 without SA, Column 2: Con A without SA, Column 3: SBA without SA, Column 4: mAb H101 with SA, Column 5: Con A with SA, Column 6: SBA with SA. Antibody and lectin concentrations in the test wells after serial dilution are as follows: Row A: 100 μg/mL, Row B: 30 μg/mL, Row C: 10 μg/mL, Row D: 3 μg/mL. Row E: 1 μg/mL Row F: 0 ng/ml.

Based on agglutination assay results, half maximal effective concentrations (EC50) were calculated for tested antibody and lectins as shown in Table 3.

TABLE 3
mAb H101 and Lectin EC50 values

| mAb/Lectin | EC50, ug/mL (w/o SA) | EC50, ug/mL (with SA) |
|---|---|---|
| H101 | <1 | <1 |
| ConA | ~100 | 30 |
| SBA | 30 | 30 |

This data demonstrates mAb H101 provides hemagglutinating activity of pooled human red blood cells (hRBC) with and without streptavidin as demonstrated by low EC50 values.

### Example 3: Evaluation of Lateral Flow Immunoassays Comprising Hemagglutination Agents

A study was performed to evaluate mAb H101 activity as a hemagglutination agent when present on a lateral flow strip test. The same mAb H101 antibody as described above in the agglutination assays of Example 2 was added to the sample receiving zone of a lateral flow strip test to analyze whether red blood cells could be trapped/retained in the sample pad when a whole blood sample is added to the sample receiving zone of a lateral flow immunoassay (strip test).

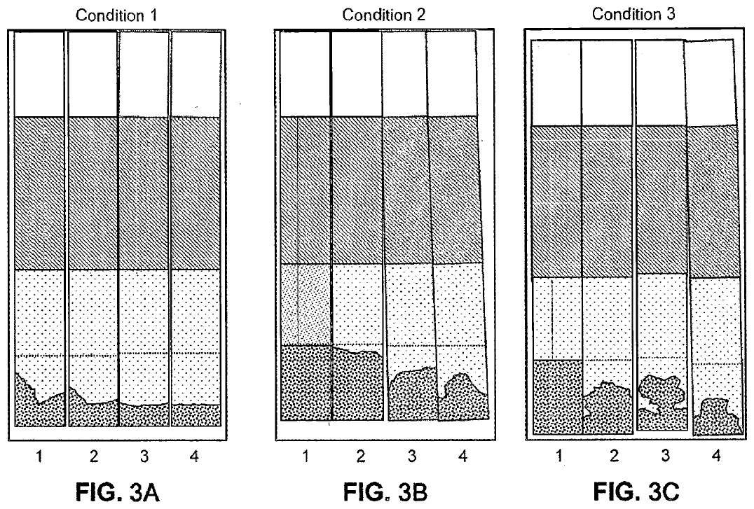

Lateral flow strip tests were assembled with three different treatments applied to the sample pad in a sample receiving zone of a lateral flow test. The sample pad treatments included Condition 1) Blank control without any antibody (FIG. 3A), Condition 2) mAb H101 antibody at 0.74 mg/mL in PBS (FIG. 3B), and Condition 3) mAb H101 antibody at 0.74 mg/mL with 1 mg/ml streptavidin in PBS (FIG. 3C). 1 mL of prepared treatment solution was added to each foot of sample pad and allowed to dry for 1 hour at room temperature.

For configurations wherein pad treatments included Condition 1) Blank control without any antibody, retention of red blood cells in the sample pad is achieved by physical means such as the red blood cells being trapped in a net-like mesh of the sample pad (FIG. 3A). For configurations wherein pad treatments included Conditions 1) and 2) including mAb H101 with or without streptavidin, retention of red blood cells in the sample pad is achieved by agglutination of red blood cells by the antibody and/or streptavidin in addition to physical means such as the agglutinated red blood cells being trapped in the net-like mesh of the sample pad (FIGS. 3B and 3C)

Various amounts of pooled human red blood cells (Rockland #R407-0050) suspended in 10% PBS solution were then pipetted onto the sample pad followed by addition of a further chase volume of 10% PBS to a final volume of 100 L. Four different red blood cell/PBS volumes were tested, as follows: 1) 50 μL RBC+50 μl PBS; 2) 25 μL RBC +75 μl PBS; 3) 12.5 μL RBC+87.5 μl PBS; 4) 6.2 μL RBC+93.8 μl PBS.

After addition of the red blood cells and chase PBS, the lateral flow strip tests were allowed to run for 10 minutes at room temperature. After 10 minutes, trapping/retention of red blood cells in the sample receiving zone was visualized as shown in FIG. 3 for conditions 1, 2 and 3 and RBC/PBS volumes 1, 2, 3 and 4.

This data demonstrates control strips retain red blood cells in the sample pad due to physical trapping (see Condition 1, FIG. 3A). Additionally, including biotinylated mAb H101 worked efficiently to trap hRBCs in the sample receiving zone with an approximate capacity of 12.5 μL RBC (see Condition 2, FIG. 3B). Moreover, in the presence of streptavidin, biotinylated mAb H101 was even more effective for trapping/retaining hRBCs in the sample receiving zone with an approximate capacity of at least 50 μL RBC (see Condition 3, FIG. 3C).

The red blood cell retention and agglutination agents, devices and methods described herein have a number of advantages. For example, the lateral flow immunoassay device containing a physical red blood cell trapping means and/or an agglutination agent in the sample receiving zone can be used to analyze whole blood samples without optical interference from red blood cells. Also, advantages of the described physical trapping and/or hemagglutinating lateral flow assays allow separation of red blood cells from a whole blood sample without any extra steps, devices, or manipulations but instead the separation occurs directly in the sample receiving zone of the lateral flow immunoassay device. This provides lab personnel, or other device users, such as over the counter at home strip test users, to quickly, easily and accurately use lateral flow strip tests for analysis of whole blood, such as blood from a finger prick, without extra complicated or time consuming steps. By further example, the described whole blood trapping/agglutinating lateral flow immunoassays require no additional equipment, providing a single-use separation and analysis device without the need for lab instruments, such as a centrifuge or the like to separate blood fractions.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

A phrase such as “an embodiment” or “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the methods and systems described herein may be embodied in a variety of other forms without departing from the spirit thereof. The accompanying claims and their equivalents are intended to cover such forms or modifications as would fall within the scope and spirit of the subject technology.

1. A device for detection of an analyte of interest in a whole blood sample, comprising:
(i) a sample receiving zone comprising a non-lytic hemagglutination agent, wherein the hemagglutination agent captures red blood cells from an applied whole blood sample in the sample receiving zone without lysis of the red blood cells,
(ii) a labeling zone comprising a means for specifically labeling the analyte of interest, and
(iii) a capture zone comprising means for specifically binding and immobilizing the labeled analyte of interest,
wherein the sample receiving zone, the labeling zone and the capture zone are arranged in a liquid flow path.

2. The device of claim 1, wherein the hemagglutination agent is an antibody.

3. The device of claim 2, wherein the antibody is a monoclonal antibody that binds human red blood cells.

4. The device of claim 2, wherein the antibody is a polyclonal antibody that binds human red blood cells.

5. The device of claim 2, wherein the antibody binds H antigen of human red blood cells.

6. The device of claim 1, wherein the hemagglutination agent is self-immobilized in the sample receiving zone.

7. The device of claim 2, wherein the antibody is biotinylated.

8. The device of claim 7, wherein the sample receiving zone further comprises streptavidin.

9. The device of claim 8, wherein the streptavidin is on particles immobilized in the sample receiving zone.

10. The device of claim 1, wherein the hemagglutination agent is a lectin.

11. The device of claim 1, wherein the hemagglutination agent is wheat germ agglutinin, Ulex europaeus agglutinin I, Pisum sativum agglutinin, Lens culinaris agglutin, or Phaseolus vulgaris erythroagglutinin.

12. The device of claim 10, wherein the lectin is self-immobilized in the sample receiving zone.

13. The device of claim 10, wherein the lectin is biotinylated.

14. The device of claim 13, wherein the sample receiving zone further comprises streptavidin.

15. The device of claim 14, wherein the streptavidin is on particles immobilized in the sample receiving zone.

16. The device of claim 1, wherein the means for specifically labeling the analyte of interest is a detectable antibody.

17-18. (canceled)

19. The device of claim 1, wherein the means for specifically labeling the analyte of interest is a detectable antigen

20-23. (canceled)

24. The device of claim 1, wherein the means for specifically binding and immobilizing the labeled analyte of interest is an immobilized antibody.

25-26. (canceled)

27. The device of claim 1, wherein the means for specifically binding and immobilizing the labeled analyte of interest is an immobilized antigen.

28-31. (canceled)

32. A device for detection of an analyte of interest in a whole blood sample, comprising:
(i) a sample receiving zone comprising a physical means for red blood cell retention, wherein the physical means captures red blood cells from an applied whole blood sample in the sample receiving zone without lysis of the red blood cells,
(ii) a labeling zone comprising a means for specifically labeling the analyte of interest, and
(iii) a capture zone comprising means for specifically binding and immobilizing the labeled analyte of interest,
wherein the sample receiving zone, the labeling zone and the capture zone are arranged in a liquid flow path.

33. The device of claim 32, wherein the physical means for red blood cell retention comprises a net, mesh, screen, or lattice with appropriate gap sizes for physically retaining and/or trapping red blood cells in the sample receiving zone.

34. The device of claim 32, wherein the sample receiving zone further comprises a hemagglutination agent.

35. The device of claim 34, wherein the hemagglutination agent is an antibody that binds human red blood cells.

36-37. (canceled)

38. The device of claim 35, wherein the antibody binds H antigen of human red blood cells.

39. The device of claim 34, wherein the hemagglutination agent is self-immobilized in the sample receiving zone.

40-42. (canceled)

43. The device of claim 34, wherein the hemagglutination agent is a lectin.

44. The device of claim 34, wherein the hemagglutination agent is wheat germ agglutinin, Ulex europaeus agglutinin I, Pisum sativum agglutinin, Lens culinaris agglutin, or Phaseolus vulgaris erythroagglutinin.

45. The device of claim 43, wherein the lectin is self-immobilized in the sample receiving zone.

46. The device of claim 34, wherein the hemagglutination agent is biotinylated.

47. The device of claim 46, wherein the sample receiving zone further comprises streptavidin.

48. The device of claim 47, wherein the streptavidin is on particles immobilized in the sample receiving zone.

49. The device of claim 32, wherein the means for specifically labeling the analyte of interest is a detectable antibody.

50-51. (canceled)

52. The device of claim 32, wherein the means for specifically labeling the analyte of interest is a detectable antigen.

53-56. (canceled)

57. The device of claim 32, wherein the means for specifically binding and immobilizing the labeled analyte of interest is an immobilized antibody.

58-59. (canceled)

60. The device of claim 32, wherein the means for specifically binding and immobilizing the labeled analyte of interest is an immobilized antigen.

61-64. (canceled)

65. A method for detecting the presence or absence of an analyte of interest in a whole blood sample, comprising:
providing a device according to claim 1,
placing a whole blood sample on the device, wherein red blood cells are retained in the sample receiving zone; and
determining the presence or absence of labeled analyte immobilized at the capture zone.

66. The method of claim 65, further comprising:
providing an instrument for collecting the whole blood sample; and collecting a whole blood sample on the instrument.

67. A kit, comprising:
a device according to claim 1;
an instrument for collecting a whole blood sample; and
instructions for use.

68. The kit of claim 67, wherein the instrument is a capillary tube.
